(19) United States
(12) Patent Application Publication
(10) Pub. No.: US 2015/0305469 A1
(43) Pub. Date: Oct. 29, 2015

(54) METHOD FOR STRAIGHTENING THE HAIR USING A COMPOSITION CONTAINING GLYOXYLIC ACID AND/OR A DERIVATIVE THEREOF

(71) Applicant: L'OREAL, Paris (FR)

(72) Inventor: Laurence PAUL, St Leu la Foret (FR)

(21) Appl. No.: 14/441,526

(22) PCT Filed: Nov. 7, 2013
(86) PCT No.: PCT/FR2013/052669

§ 371 Date: May 8, 2015

(30) Foreign Application Priority Data
Nov. 9, 2012 (FR) 1260660

(51) Int. Cl. A45D 7/06; A45D 2/00; A61K 8/891; A61Q 5/06; A61K 8/365

(52) U.S. Cl. CPC A45D 7/06; A61Q 5/06; A61K 8/365; A61K 8/891; A45D 2/001; A61K 2800/884; A61K 2800/592

(57) ABSTRACT

The subject matter of the present invention is a method for straightening/relaxing the hair, which comprises (i) the application to the hair of a cosmetic composition comprising at least glyoxylic acid and/or at least one derivative thereof, the composition having a pH of less than or equal to 4, (ii) a leave on time for the composition comprising glyoxylic acid and/or at least one derivative thereof of at least 10 min, followed by (iii) the application, without rinsing, of a composition comprising at least one hair treatment compound chosen from fatty alcohols and cationic conditioning agents, and then by (iv) a heat application step comprising at least one step of heating at a temperature of at least 150° C.

The method for straightening the hair according to the invention is effective in terms of straightening and quickly implemented.

The present invention relates to a particular method for straightening keratin fibers, in particular the hair, using a composition comprising glyoxylic acid and/or a derivative thereof.

In the hair field, consumers wish to have available compositions which make it possible to introduce a temporary change to their head of hair, while targeting good persistence of the effect produced. In general, it is desired for the change to withstand shampooing for at least fifteen days or even more, depending on the nature of said change.

Treatments already exist for modifying the color or shape of the hair and also, to a certain extent, the texture of the hair. One of the treatments known for modifying the texture of the hair consists in combining heat and a composition comprising formaldehyde. This treatment is in particular effective for imparting a better appearance to damaged hair and/or for treating long hair and curly hair.

The action of formaldehyde is associated with its ability to crosslink proteins by reaction on the nucleophilic sites thereof. The heat used may be that of an iron (flat tongs or crimping iron), the temperature of which may generally be up to 200° C. or more. However, it is increasingly sought to avoid the use of such substances, which may prove to be aggressive to the hair and other keratin materials.

Patent application WO 2011/104 282 thus proposed a novel method for semi-permanently straightening the hair, which consists in applying a solution which may contain an a-keto acid to the hair for 15 to 120 minutes, then drying and, finally, straightening the head of hair with an iron at a temperature of about 200° C. The α-keto acid employed is preferably glyoxylic acid.

However, it has been noted that the use of these a-keto acids, such as glyoxylic acid, may create some important limitations; in particular, at strong concentration, they may not be well tolerated, in particular when the scalp is sensitive and/or irritated. Their volatility, amplified by the use of heat, for example by means of a crimping iron or a straightening iron, may also pose a problem. Moreover, cosmetic formulations at acid pH may degrade the hair and/or modify the color thereof.

Moreover, this type of straightening method is often associated with various steps of washing, drying, conditioning, and hair shaping with a brush or with a hairdryer, which make these methods laborious to carry out, in particular in terms of hair treatment time to obtain the desired hair straightening.

Finally, straightening methods which use an iron are often accompanied by the giving off of fumes, which can be detrimental to the comfort of the person using the composition and of the stylist who applies it.

The aim of the invention is to develop a novel method for rapid straightening/relaxing of the hair which makes it possible to straighten/relax and/or reduce the volume of the hair efficiently and in a persistent manner while limiting the degradation of the hair, while at the same time maintaining comfort at the time of application for the user of the composition, but also for the stylist who applies it.

Thus, a subject of the present invention is a method for straightening/relaxing keratin fibers, in particular the hair, which comprises (i) the application to said fibers of a cosmetic composition comprising at least glyoxylic acid and/or a derivative thereof, the composition having a pH of less than or equal to 4, (ii) a leave-on time for the composition comprising glyoxylic acid and/or a derivative thereof of at least 10 min, preferably ranging from 10 to 60 min, followed by (iii) the application, without rinsing, of a composition comprising at least one hair treatment compound chosen from fatty alcohols and cationic conditioning agents, and then by (iv) a heat application step comprising at least one step of heating at a temperature of at least 150° C.

Good straightening of the keratin fibers with limited degradation of these keratin fibers is obtained with the method of the invention, even when the application of the composition is followed by a heat treatment, in particular by means of a hair straightening iron. It will also make it possible to obtain satisfactory temporary straightening of the hair while at the same time maintaining quick implementation of the method. Hair straightening which limits the degradation of the physical properties of the hair, while at the same time reducing the frizziness effect in a long-lasting manner, is thus obtained in a short time. The comfort both for the use of the composition and for the stylist who applies it is satisfactory.

In that which follows, the expression “at least one” is equivalent to the expression “one or more”.

Preferably, the composition according to the invention does not comprise any coloring agent or any reducing agent.

According to the present invention, the term “coloring agents” is intended to mean agents for coloring keratin fibers, such as direct dyes, pigments or oxidation dye precursors (bases and couplers). If they are present, their content does not exceed 0.001% by weight relative to the total weight of the composition. Specifically, at such a content, only the composition would be dyed, i.e. no dyeing effect would be observed on the keratin fibers.

It is recalled that oxidation dye precursors, oxidation bases and couplers are colorless or sparingly colored compounds, which, via a condensation reaction in the presence of an oxidizing agent, give a colored species. With regard to direct dyes, these compounds are colored and have a certain affinity for keratin fibers.

According to the present invention, the term “reducing agent” is intended to mean an agent that is capable of reducing the disulfide bonds of the hair, such as compounds chosen from thiols, alkaline sulfites, hydrides and phosphines.

In the present invention, the glyoxylic acid and/or derivatives thereof may be in free form, in salt form, but also in the hydrate forms thereof. As glyoxylic acid derivatives, mention may be made of glyoxylic acid esters, glyoxylic acid amides and glyoxylic acid acetals and hemiacetals. The glyoxylic acid esters are, for example, obtained from glyoxylic acid and a mono or polyalcohol.

The term “mono or polyalcohol” is intended to mean an organic compound comprising one hydroxyl group (monoalcohol) or at least two hydroxyl groups (polyalcohol or polyol); said hydroxylated organic compound possibly being aliphatic, acyclic, linear or branched, or (hetero)cyclic, such as sugars (monosaccharides or polysaccharides) or sugar alcohols. More particularly, the polyalcohol comprises from 2 to 100 hydroxyl groups, preferentially from 2 to 20 hydroxyl groups, even more preferentially from 2 to 10 hydroxyl groups, and better still 2 or 3 hydroxyl groups. Preferably, the mono or polyalcohol is chosen from methanol, ethanol, propanol, isopropanol, butanol, hexanol, ethylene glycol, glycerol, dihydroxyacetone, glucose, sorbitol and menthol.

By way of esters, mention may particular be made of methyl glyoxylate, ethyl glyoxylate, glyceryl glyoxylate, dihydroxyacetone glyoxylate, glyceryl diglyoxylate or triglyoxylate, sorbitol mono-, di- or triglyoxylate, glucose mono-, di- or triglyoxylate, menthyl glyoxylate, and acetals, hemiacetals and hydrates thereof.

The glyoxylic acid amides are, for example, obtained from glyoxylic acid and an organic mono or polyamine.

The term “mono or polyamine” is intended to mean an organic compound comprising one amino group (monoamine) or at least two (and preferably from 2 to 100, better still from 2 to 20) amine groups; said organic compound possibly being aliphatic, acyclic, linear or branched or (hetero)cyclic. The term “amino group” is intended to mean a primary amine group —NH₂, or a secondary amine group >NH.

The mono or polyamine is preferably aliphatic. This amine is preferably chosen from methylamine, ethylamine, propylamine, isopropylamine, butylamine, hexylamine, monoethanolamine, monopropanolamine, propane-1,2,3-triamine and diaminoacetone. Mention may in particular be made of glyoxylic acid N-beta-hydroxyethylamide and glyoxylic acid N-gamma-hydroxypropylamide, and acetals, hemiacetals and/or hydrates thereof.

The glyoxylic acid acetals and hemiacetals may, for example, be obtained from the reaction of alcohols with blocked forms of glyoxylic acid and then hydrolysis. The alcohols may be the same as those mentioned for the esters. The acetals may also be cyclic acetals. Mention may in particular be made of dimethoxyacetic acid, diethoxyacetic acid, 1,3-dioxane-2-carboxylic acid and 1,3-dioxolane-2-carboxylic acid.

The salts may be salts resulting from the interaction of the compounds of formula (I) with acids or bases, it being possible for the acids or bases to be of organic or inorganic nature.

Preferably, the salts are salts resulting from the interaction of the compounds of formula (I) with bases. Alkali metal or alkaline-earth metal salts, and in particular the sodium salts, will in particular be mentioned.

Preferably, the glyoxylic acid and/or derivatives thereof are used in free (non-salified) form or in a hydrate form.

Thus, the method of the present invention is neither carried out with a step of permanent reshaping at a basic pH, nor based on a reducing agent. The compositions according to the invention, and in particular the one comprising glyoxylic acid and/or derivatives thereof, may be in any of the galenic forms conventionally used, and in particular in the form of an aqueous, alcoholic or aqueous-alcoholic, or oily solution or suspension; a solution or a dispersion of the lotion or serum type; an emulsion, in particular of liquid or semi-liquid consistency, of the O/W, W/O or multiple type; a suspension or emulsion of soft consistency of cream (O/W) or (W/O) type; an aqueous or anhydrous gel, or any other cosmetic form.

According to one embodiment of the method of the invention, the composition containing glyoxylic acid and/or derivatives thereof is aqueous or anhydrous. It is preferably aqueous and then comprises water at a concentration ranging from 5% to 98%, better still from 5% to 50% and even better still from 10% to 40% by weight relative to the total weight of the composition.

According to one embodiment, the composition of the invention is in the form of an aqueous composition comprising from 0.1% to 20% of glyoxylic acid and/or derivatives thereof, preferably at least 3% of glyoxylic acid and/or derivatives thereof, preferentially from 3% to 10% by weight of the total weight of the composition.

The pH of the composition is less than 4 and preferably ranges from 1 to 3, better still from 1.7 to 3.

The composition containing glyoxylic acid and/or a derivative thereof is preferably in the form of gels, lotions or creams, masks or sera.

The method of the invention comprises a step (ii) of leaving the previously described composition on the hair. This leave-on time generally ranges from 10 to 60 min and preferably from 20 to 35 min.

In step (iii), and without rinsing off the composition containing glyoxylic acid and/or at least one derivative thereof, a composition comprising at least one hair treatment compound chosen from fatty alcohols and/or cationic conditioning agents, preferably an amount ranging from 0.01% to 10% by weight, even better still between 0.1% and 8%, and even more preferentially from 0.5% to 5% by weight, relative to the total weight of the composition containing it or them, is applied. By way of example of cationic conditioning agents, mention may be made of cationic polymers and cationic surfactants. These compounds may be contained in a composition comprising other cosmetic ingredients. According to this embodiment, the composition applied in step (iii) is free of glyoxylic acid and/or of a derivative thereof.

The term “fatty alcohol” is intended to mean a long-chain aliphatic alcohol comprising from 8 to 40 carbon atoms and comprising at least one hydroxyl group OH. These fatty alcohols are neither oxyalkylenated nor glycerolated. Preferably, the solid fatty alcohols have the structure R—OH with R denoting a linear or branched alkyl or alkenyl group, optionally substituted with one or more hydroxyl groups, comprising from 8 to 40, better still from 10 to 30, or even from 12 to 24 and even better still from 14 to 22 carbon atoms.

Preferably, the fatty alcohols of the invention are not liquid at ambient temperature (25° C.) and at atmospheric pressure (1.013×10⁵ Pa; 760 mmHg). Even more preferentially, they are solid under these conditions.

Preferably, R denotes a linear alkyl group, optionally substituted with one or more hydroxyl groups, comprising from 8 to 40, better still from 10 to 30, or even from 12 to 24 and even better still from 14 to 22 carbon atoms.

The fatty alcohols that can be used may be chosen from, alone or as a mixture:
lauryl alcohol (1-dodecanol);
myristyl alcohol (1-tetradecanol);
cetyl alcohol (1-hexadecanol);
stearyl alcohol (1-octadecanol);
arachidyl alcohol (1-eicosanol);
behenyl alcohol (1-docosanol);
lignoceryl alcohol (1-tetracosanol);
ceryl alcohol (1-hexacosanol);
montanyl alcohol (1-octacosanol);
myricyl alcohol (1-triacontanol).

Preferentially, the fatty alcohol is chosen from cetyl alcohol, stearyl alcohol, behenyl alcohol and mixtures thereof such as cetylstearyl alcohol or cetearyl alcohol. The fatty alcohols may be mixed, which means that several species, in particular of different chain lengths, may coexist in a commercial product, in the form of a mixture.

The cationic surfactants which can be used in step (iii) are well known per se; for example, mention may be made of optionally polyoxyalkylenated primary, secondary or tertiary fatty amine salts, quaternary ammonium salts, and mixtures thereof.

Examples of quaternary ammonium salts that may in particular be mentioned include:
those that have the general formula (I) below:

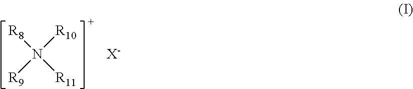

in which the radicals R₈ to R₁₁, which may be identical or different, represent a linear or branched aliphatic radical comprising from 1 to 30 carbon atoms or an aromatic radical such as aryl or alkylaryl, at least one of the radicals R₈ to R₁₁ comprising from 8 to 30 carbon atoms, or an aromatic radical such as aryl or alkylaryl. The aliphatic radicals may comprise heteroatoms such as, in particular, oxygen, nitrogen, sulfur and halogens. The aliphatic radicals are chosen, for example, from alkyl, alkoxy, polyoxyalkylene (C₂-C₆), alkylamide, (C₁₂C₂₂)alkylamido(C₂-C₆)alkyl, (C₁₂-C₂₂)alkyl acetate and hydroxyalkyl radicals containing approximately from 1 to 30 carbon atoms; X is an anion chosen from the group of halides, phosphates, acetates, lactates, (C₂-C₆)alkyl sulfates and alkyl or alkylaryl sulfonates;
quaternary ammonium salts of imidazoline, for instance those of formula (II) below:

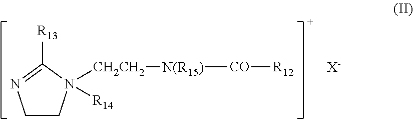

in which R₁₂ represents an alkenyl or alkyl radical comprising from 8 to 30 carbon atoms, for example derived from tallow fatty acids, R₁₃ represents a hydrogen atom, a C₁-C₄ alkyl radical or an alkenyl or alkyl radical comprising from 8 to 30 carbon atoms, R₁₄ represents a C₁-C₄ alkyl radical, R₁₅ represents a hydrogen atom or a C₁-C₄ alkyl radical and X⁻ is an anion chosen from the group of the halides, phosphates, acetates, lactates, alkyl sulfates, and alkyl or alkylaryl sulfonates. Preferably, R₁₂ and R₁₃ denote a mixture of alkenyl or alkyl radicals comprising from 12 to 21 carbon atoms, for example derived from tallow fatty acids, R₁₄ denotes a methyl radical and R₁₅ denotes a hydrogen atom. Such a product is sold, for example, under the name Rewoquat® W 75 by the company Rewo;
diquaternary ammonium salts of formula (III):

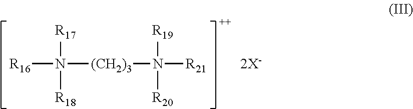

in which R₁₆ denotes an aliphatic radical containing from about 16 to 30 carbon atoms, R₁₇, R₁₈, R₁₉, R₂₀ and R₂₁, which may be identical or different, are chosen from hydrogen or an alkyl radical containing from 1 to 4 carbon atoms, and X is an anion chosen from the group of halides, acetates, phosphates, nitrates and methyl sulfates. Such diquaternary ammonium salts in particular comprise propanetallowdiammonium dichloride;
quaternary ammonium salts containing at least one ester function, such as those of formula (IV) below:

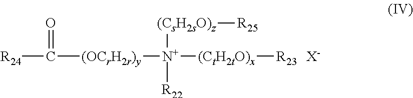

in which:

R₂₂ is chosen from C₁-C₆ alkyl radicals and C₁-C₆ hydroxyalkyl or dihydroxyalkyl radicals;

R₂₃ is chosen from:
the radical

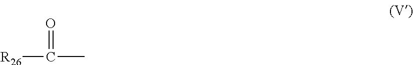

linear or branched, saturated or unsaturated C₁-C₂₂ hydrocarbon-based radicals R₂₇,
a hydrogen atom,

R₂₅ is chosen from:
the radical

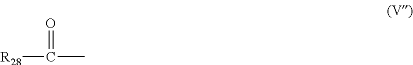

linear or branched, saturated or unsaturated C₁-C₆ hydrocarbon-based radicals R₂₉,
a hydrogen atom,

R₂₄, R₂₆ and R₂₈, which are identical or different, are chosen from linear or branched, saturated or unsaturated C₇-C₂₁ hydrocarbon-based radicals;

r, s and t, which are identical or different, are integers having values from 2 to 6;

y is an integer having a value from 1 to 10;

x and z, which may be identical or different, are integers ranging from 0 to 10;

X⁻ is a simple or complex, organic or inorganic anion;

with the proviso that the sum x+y+z is from 1 to 15, that when x is 0 then R₂₃ denotes R₂₇, and that when z is 0 then R₂₅ denotes R₂₉.

The alkyl radicals R₂₂ may be linear or branched, and more particularly linear. Preferably, R₂₂ denotes a methyl, ethyl, hydroxyethyl or dihydroxypropyl radical and more particularly a methyl or ethyl radical.

Advantageously, the sum x+y+z is from 1 to 10.

When R₂₃ is a hydrocarbon-based radical R₂₇, it may be long and have from 12 to 22 carbon atoms, or may be short and have from 1 to 3 carbon atoms.

When R₂₅ is a hydrocarbon-based radical R₂₉, it has preferably 1 to 3 carbon atoms.

Advantageously, R₂₄, R₂₆ and R₂₈, which are identical or different, are chosen from linear or branched, saturated or unsaturated C₁₁-C₂₁ hydrocarbon-based radicals, and more particularly from linear or branched, saturated or unsaturated C₁₁-C₂₁ alkyl and alkenyl radicals.

Preferably, x and z, which may be identical or different, are equal to 0 or 1. Advantageously, y is equal to 1.

Preferably, r, s and t, which may be identical or different, are equal to 2 or 3, and even more particularly are equal to 2.

The anion is preferably a halide (chloride, bromide or iodide) or an alkyl sulfate, more particularly methyl sulfate. However, it is possible to use methanesulfonate, phosphate, nitrate, tosylate, an anion derived from an organic acid, such as acetate or lactate, or any other anion that is compatible with the ammonium containing an ester function.

The anion X⁻ is even more particularly chloride or methyl sulfate.

Use is made more particularly, in the composition according to the invention, of the ammonium salts of formula (IV) in which:
R₂₂ denotes a methyl or ethyl radical;
x and y are equal to 1;
z is equal to 0 or 1;
r, s and t are equal to 2;
R₂₃ is chosen from:
   the radical

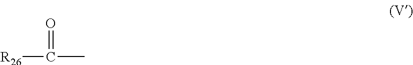

methyl, ethyl or C₁₄-C₂₂ hydrocarbon-based radicals;
a hydrogen atom;
R₂₅ is chosen from:
   the radical

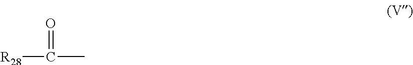

a hydrogen atom;
R₂₄, R₂₆ and R₂₈, which are identical or different, are chosen from linear or branched, saturated or unsaturated C₁₃-C₁₇ hydrocarbon-based radicals and preferably from linear or branched, saturated or unsaturated C₁₃-C₁₇ alkyl and alkenyl radicals.

The hydrocarbon-based radicals are advantageously linear.

Mention may be made, for example, of compounds of formula (IV), such as diacyloxyethyldimethylammonium, diacyloxyethyl(hydroxyethyl)methylammonium, monoacyloxyethyldi(hydroxyethyl)methylammonium, triacyloxyethylmethylammonium or monoacyloxyethyl(hydroxyethyl)dimethylammonium salts (in particular chloride or methyl sulfate), and mixtures thereof. The acyl radicals preferably contain 14 to 18 carbon atoms and are obtained more particularly from a plant oil such as palm oil or sunflower oil. When the compound contains several acyl radicals, these radicals may be identical or different.

These products are obtained, for example, by direct esterification of triethanolamine, triisopropanolamine, alkyldiethanolamine or alkyldiisopropanolamine, which are optionally oxyalkylenated, with fatty acids or with mixtures of fatty acids of plant or animal origin, or by transesterification of the methyl esters thereof. This esterification is followed by a quaternization using an alkylating agent such as an alkyl halide (preferably a methyl or ethyl halide), a dialkyl sulfate (preferably a dimethyl or diethyl sulfate), methyl methanesulfonate, methyl para-toluenesulfonate, glycol chlorohydrin or glycerol chlorohydrin.

Such compounds are, for example, sold under the names Dehyquart® by the company Henkel, Stepanquat® by the company Stepan, Noxamium® by the company Ceca or Rewoquat® WE 18 by the company Rewo-Witco. The composition according to the invention preferably contains a mixture of quaternary ammonium monoester, diester and triester salts with a weight majority of diester salts.

Mixtures of ammonium salts that can be used include, for example, the mixture containing 15% to 30% by weight of acyloxyethyldihydroxyethylmethylammonium methyl sulfate, 45% to 60% of diacyloxyethylhydroxyethylmethylammonium methyl sulfate and 15% to 30% of triacyloxyethylmethylammonium methyl sulfate, the acyl radicals having from 14 to 18 carbon atoms and originating from palm oil, which is optionally partially hydrogenated.

Use may also be made of the ammonium salts containing at least one ester function that are described in patents U.S. Pat. No. 4,874,554 and U.S. Pat. No. 4,137,180.

Among the quaternary ammonium salts, preference is in particular given to those which correspond to formula (I) in which:
R₈ represents a C₁₂-C₃₀, preferably C₁₄-C₂₂, alkyl group, a C₁₂-C₃₀ alkenyl group, a (C₁₂-C₂₂)alkylamido(C₂-C₆)alkyl group, a (C₁₂-C₂₂)alkyl acetate group, or an aromatic group that such as C₆-C₁₂ aryl or alkylaryl,
R₉ to R₁₁, which may be identical or different, represent a C₁-C₈ alkyl, C₁-C₈ alkenyl, C₁-C₈ alkoxy, C₁-C₈ hydroxyalkyl, (C₂-C₆) polyoxyalkylene or C₁-C₈ alkylamide group; and
X is an anion chosen from the group of halides, phosphates, acetates, lactates, (C₂-C₆)alkyl sulfates and alkyl or alkylaryl sulfonates.

By way of examples of cationic surfactants that are particularly preferred, mention may in particular be made, on the one hand, of tetraalkylammonium salts, in particular tetraalkylammonium chlorides, for instance dialkyldimethylammonium or alkyltrimethylammonium chlorides in which the alkyl radical comprises approximately from 12 to 30 carbon atoms, in particular distearyldimethylammonium chloride, behenyltrimethylammonium chloride, arachidyltrimethylammonium chloride, stearyltrimethylammonium chloride, cetyltrimethylammonium chloride and benzyldimethylstearylammonium chloride or else, on the other hand, (C₈-C₃₀)alkylamido(C₂-C₆)alkyltrimethylammonium salts, in particular palmitylamidopropyltrimethylammonium chloride or stearamidopropyldimethyl-(myristyl acétate)-ammonium chloride sold under the name Ceraphyl® 70 by the company Van Dyk, and dipalmitoylethylhydroxyethylmethylammonium methosulfate.

The cationic surfactants that are particularly preferred are chosen in particular from behenyltrimethylammonium chloride, cetyltrimethylammonium chloride, palmitylamidopropyltrimethylammonium chloride, and dipalmitoylethylhydroxyethylmethylammonium methosulfate.

The composition applied to the keratin fibers preferably comprises the cationic surfactant(s) in an amount ranging from 0.01% to 10% by weight, even better still between 0.1% and 8% by weight, and even more preferentially from 0.5% to 5% by weight, relative to the total weight of the composition containing it or them.

By way of cationic polymer, mention may be made of polymers comprising primary, secondary, tertiary and/or quaternary amine groups forming part of the polymer chain or directly attached thereto, and having a molecular weight of between 500 and about 5 000 000 and preferably between 1000 and 3 000 000.

Among the cationic polymers, mention may more particularly be made of polymers of the polyamine, polyaminoamide and polyquaternary ammonium type. These are known products. They are described, for example, in French patents 2 505 348 and 2 542 997.

Among these polymers, mention may be made of:
(1) homopolymers or copolymers derived from acrylic or methacrylic esters or amides and comprising at least one of the units of following formulae:

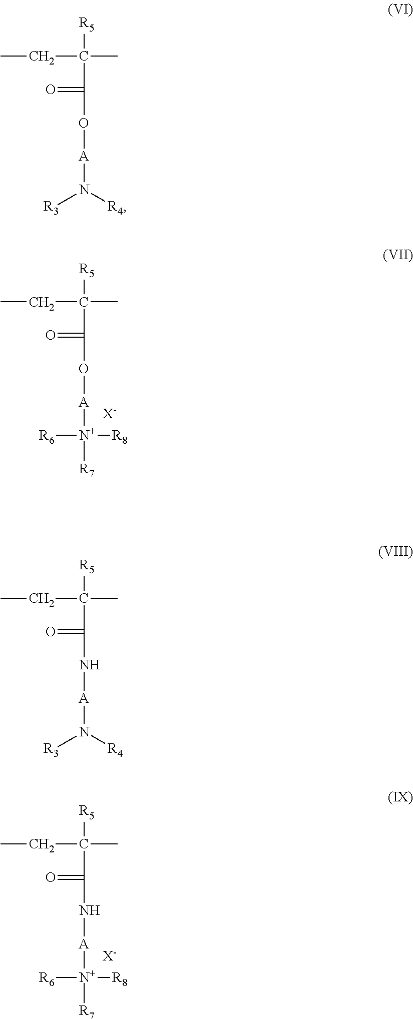

in which:
R₃ and R₄, which may be identical or different, represent hydrogen or an alkyl group containing from 1 to 6 carbon atoms and preferably methyl or ethyl;
R₅, which may be identical or different, denote a hydrogen atom or a CH₃ radical;
A, which may be identical or different, represent a linear or branched alkyl group of 1 to 6 carbon atoms, preferably 2 or 3 carbon atoms, or a hydroxyalkyl group of 1 to 4 carbon atoms;
R₆, R₇ and R₈, which may be identical or different, represent an alkyl group containing from 1 to 18 carbon atoms or a benzyl radical and preferably an alkyl group containing from 1 to 6 carbon atoms;
X denotes an anion derived from an inorganic or organic acid, such as a methosulfate anion or a halide such as chloride or bromide.

The copolymers of family (1) may also contain one or more units derived from comonomers that may be chosen from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Thus, among these copolymers of the family (1), mention may be made of:
copolymers of acrylamide and of dimethylaminoethyl methacrylate quaternized with dimethyl sulfate or with a dimethyl halide, such as the product sold under the name Hercofloc by the company Hercules,
copolymers of acrylamide and methacryloyloxyethyltrimethylammonium chloride, described for example in patent application EP-A-080976 and sold under the name Bina Quat P 100 by the company Ciba Geigy,
the copolymer of acrylamide and of methacryloyloxyethyltrimethylammonium methosulfate sold under the name Reten by the company Hercules,
quaternized or non-quaternized vinylpyrrolidone/dialkylaminoalkyl acrylate or methacrylate copolymers, such as the products sold under the name Gafquat by the company ISP, such as, for example, Gafquat 734 or Gafquat 755, or alternatively the products known as Copolymer 845, 958 and 937. These polymers are described in detail in French patents 2 077 143 and 2 393 573,
dimethylaminoethyl methacrylate/vinylcaprolactam/vinylpyrrolidone terpolymers, such as the product sold under the name Gaffix VC 713 by the company ISP,
vinylpyrrolidone/methacrylamidopropyldimethylamine copolymers sold in particular under the name Styleze CC 10 by ISP,
quaternized vinylpyrrolidone/dimethylaminopropylmethacrylamide copolymers such as the product sold under the name Gafquat HS 100 by the company ISP, and
crosslinked polymers of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homopolymerization or copolymerization being followed by crosslinking with an olefinically unsaturated compound, in particular methylenebisacrylamide. Use may more particularly be made of a crosslinked acrylamide/methacryloyloxyethyltrimethylammonium chloride (20/80 by weight) copolymer in the form of a dispersion comprising 50% by weight of said copolymer in mineral oil. This dispersion is sold under the name Salcare® SC 92 by the company Ciba. A crosslinked methacryloyloxyethyltrimethylammonium chloride homopolymer, for example as a dispersion in mineral oil or in a liquid ester, can also be used. These dispersions are sold under the names of Salcare® SC 95 and Salcare® SC 96 by the company Ciba;
(2) polymers consisting of piperazinyl units and of divalent alkylene or hydroxyalkylene radicals containing straight or branched chains, optionally interrupted by oxygen, sulfur or nitrogen atoms or by aromatic or heterocyclic rings, and also the oxidation and/or quaternization products of these polymers. Such polymers are described, in particular, in French patents 2 162 025 and 2 280 361;
(3) water-soluble polyaminoamides prepared in particular by polycondensation of an acidic compound with a polyamine; these polyaminoamides can be crosslinked with an epihalohydrin, a diepoxide, a dianhydride, an unsaturated dianhydride, a bisunsaturated derivative, a bis-halohydrin, a bisazetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyaminoamide; these polyaminoamides can be alkylated or, if they comprise one or more tertiary amine functions, they can be quaternized. Such polymers are in particular described in French patents 2 252 840 and 2 368 508;
(4) polyaminoamide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical comprises from 1 to 4 carbon atoms and preferably denotes methyl, ethyl or propyl. Such polymers are in particular described in French patent 1 583 363.

Among these derivatives, mention may be made more particularly of the adipic acid/dimethylaminohydroxypropyl/diethylenetriamine polymers sold under the name Cartaretine F, F4 or F8 by the company Sandoz;
(5) polymers obtained by reaction of a polyalkylene polyamine containing two primary amine groups and at least one secondary amine group with a dicarboxylic acid chosen from diglycolic acid and saturated aliphatic dicarboxylic acids containing from 3 to 8 carbon atoms. The mole ratio between the polyalkylene polyamine and the dicarboxylic acid is between 0.8:1 and 1.4:1; the polyaminoamide resulting therefrom is reacted with epichlorohydrin in a mole ratio of epichlorohydrin relative to the secondary amine group of the polyaminoamide of between 0.5:1 and 1.8:1. Such polymers are described in particular in U.S. Pat. Nos. 3,227,615 and 2,961,347.

Polymers of this type are sold in particular under the name Hercosett 57 by the company Hercules Inc. or alternatively under the name PD 170 or Delsette 101 by the company Hercules in the case of the adipic acid/epoxypropyl/diethylenetriamine copolymer;
(6) cyclopolymers of alkyldiallylamine or of dialkyldiallylammonium, such as the homopolymers or copolymers containing, as main constituent of the chain, units corresponding to formula (X) or (XI):

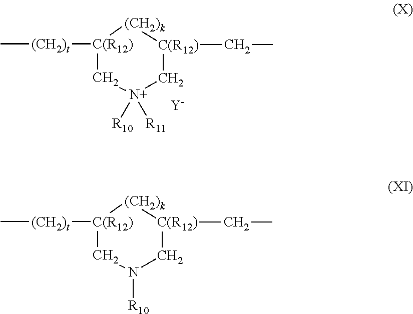

in which k and t are equal to 0 or 1, the sum k+t being equal to 1; R₁₂ denotes a hydrogen atom or a methyl radical; R₁₀ and R₁₁, independently of one another, denote an alkyl group having from 1 to 6 carbon atoms, a hydroxyalkyl group in which the alkyl group has preferably 1 to 5 carbon atoms, a lower (C₁-C₄) amidoalkyl group, or R₁₀ and R₁₁ may denote, jointly with the nitrogen atom to which they are attached, heterocyclic groups, such as piperidinyl or morpholinyl; Y⁻ is an anion such as bromide, chloride, acetate, borate, citrate, tartrate, bisulfate, bisulfite, sulfate or phosphate. These polymers are in particular described in French patent 2 080 759 and in its Certificate of Addition 2 190 406.
R₁₀ and R₁₁, independently of one another, preferably denote an alkyl group containing from 1 to 4 carbon atoms.

Among the polymers defined above, mention may be made more particularly of the dimethyldiallylammonium chloride homopolymer sold under the name Merquat 100 by the company Nalco (and homologs thereof of low weight-average molecular weights) and the copolymers of diallyldimethylammonium chloride and of acrylamide, sold under the name Merquat 550;
(7) the quaternary diammonium polymer containing repeating units corresponding to the formula:

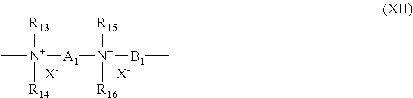

in which formula (XII):
R₁₃, R₁₄, R₁₅ and R₁₆, which may be identical or different, represent aliphatic, alicyclic or arylaliphatic radicals containing from 1 to 20 carbon atoms or lower hydroxyalkylaliphatic radicals, or alternatively R₁₃, R₁₄, R₁₅ and R₁₆, together or separately, constitute, with the nitrogen atoms to which they are attached, heterocycles optionally containing a second heteroatom other than nitrogen, or alternatively R₁₃, R₁₄, R₁₅ and R₁₆ represent a linear or branched C₁-C₆ alkyl radical substituted with a nitrile, ester, acyl or amide group or a group —C(O)—O—R₁₇-D or —C(O)—N(H)—R₁₇-D where R₁₇ is an alkylene and D is a quaternary ammonium group;
A₁ and B₁ represent polymethylene groups containing from 2 to 20 carbon atoms which may be linear or branched, and saturated or unsaturated, and which may contain, linked to or inserted in the main chain, one or more aromatic rings, or one or more oxygen or sulfur atoms or sulfoxide, sulfone, disulfide, amino, alkylamino, hydroxyl, quaternary ammonium, ureido, amide or ester groups, and
X⁻, which very identical different, denotes an anion derived from an inorganic or organic acid;
A₁, R₁₃ and R₁₅ can form, with the two nitrogen atoms to which they are attached, a piperazine ring; in addition, if A1 denotes a linear or branched, saturated or unsaturated alkylene or hydroxyalkylene radical, B1 can also denote a group —(CH₂)ₙ—C(O)-D-C(O)—(CH₂)ₚ—
n and p are integers ranging from 2 to 20 approximately
   in which D denotes:
   a) a glycol residue of formula: —O—Z—O—, where Z denotes a linear or branched hydrocarbon-based radical or a group corresponding to one of the following formulae:

—(CH₂—CH₂—O)ₓ—CH₂—CH₂—

—[CH₂—CH(CH₃)—O]_{y}—CH₂—CH(CH₃)—,
where x and y denote an integer from 1 to 4, representing a defined and unique degree of polymerization, or any number from 1 to 4, representing an average degree of polymerization;
b) a bis-secondary diamine residue, such as a piperazine derivative;
c) a bis-primary diamine residue of formula: —N(H)—Y—N(H)—, where Y denotes a linear or branched hydrocarbon radical, or alternatively the divalent radical

—CH₂—CH₂—S—S—CH₂—CH₂—;
d) a ureylene group of formula: —N(H)—C(O)—N(H)—;

preferably, X⁻ is an anion such as chloride or bromide.

These polymers have a number-average molecular weight generally of between 1000 and 100 000.

Polymers of this type are in particular described in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Use may be made more particularly of polymers that are composed of repeating units corresponding to the formula:

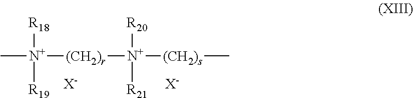

in which R₁₈, R₁₉, R₂₀ and R₂₁, which may be identical or different, denote an alkyl or hydroxyalkyl radical containing from 1 to 4 carbon atoms approximately, r and s are integers ranging from 2 to 20 approximately, and X⁻, which may be identical or different, is an anion derived from an inorganic or organic acid.

One particularly preferred compound of formula (XIII) is that for which R₁₈, R₁₉, R₂₀ and R₂₁ represent a methyl radical and r=3, s=6 and X═Cl, which is called Hexadimethrine chloride according to INCI nomenclature (CTFA);
(8) polyquaternary ammonium polymers composed of units of formula (XIV):

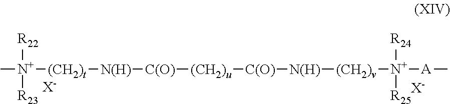

in which formula:
R₂₂, R₂₃, R₂₄ and R₂₅, which may be identical or different, represent a hydrogen atom or a methyl, ethyl, propyl, β-hydroxyethyl, β-hydroxypropyl or —CH₂CH₂(OCH₂CH₂)ₚOH radical,
where p is equal to 0 or to an integer between 1 and 6, with the proviso that R₂₂, R₂₃, R₂₄ and R₂₅ do not simultaneously represent a hydrogen atom,
t and u, which may be identical or different, are integers between 1 and 6,
v is equal to 0 or to an integer between 1 and 34,
X⁻, which may be identical or different, denotes an anion such as a halide,
A denotes a dihalide radical or preferably represents —CH₂—CH₂—O—CH₂—CH₂—.

Such compounds are described in particular in patent application EP-A-122 324.

Among these, mention may be made, for example, of the products Mirapol® A 15, Mirapol® AD1, Mirapol® AZ1 and Mirapol® 175, sold by the company Miranol.
(9) Quaternary polymers of vinylpyrrolidone and of vinylimidazole, for instance the products sold under the names Luviquat® FC 905, FC 550 and FC 370 by the company BASF;
(10) cationic polysaccharides, in particular cationic celluloses and galactomannan gums.

Among the cationic polysaccharides, mention may be made more particularly of cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups are described in French patent 1 492 597. These polymers are also defined in the CTFA dictionary as quaternary ammoniums of hydroxyethylcellulose that have reacted with an epoxide substituted with a trimethylammonium group.

Cationic cellulose copolymers or cellulose derivatives grafted with a water-soluble monomer of quaternary ammonium are described in particular in U.S. Pat. No. 4,131,576, such as hydroxyalkyl celluloses, for instance hydroxymethyl-, hydroxyethyl- or hydroxypropylcelluloses grafted, in particular, with a methacryloylethyltrimethylammonium, methacrylamidopropyltrimethylammonium or dimethyldiallylammonium salt.

The cationic galactomannan gums are described more particularly in U.S. Pat. Nos. 3,589,578 and 4,031,307, in particular guar gums containing cationic trialkylammonium groups. Guar gums modified by a 2,3-epoxypropyltrimethylammonium salt (e.g. chloride) are used, for example.

Other cationic polymers that may be used in the context of the invention are cationic proteins or cationic protein hydrolysates, polyalkyleneimines, in particular polyethyleneimines, polymers containing vinylpyridine or vinylpyridinium units, condensates of polyamines and of epichlorohydrin, quaternary polyureylenes and chitin derivatives.

The cationic proteins or protein hydrolysates are, in particular, chemically modified polypeptides bearing quaternary ammonium groups at the end of the chain, or grafted thereon. Their molecular weight may vary, for example, from 1500 to 10 000 and in particular from 2000 to 5000 approximately. Among these compounds, mention may be made in particular of:
collagen hydrolysates carrying triethylammonium groups, such as the products sold under the name Quat-Pro E by the company Maybrook and referred to in the CTFA dictionary as Triethonium Hydrolyzed Collagen Ethosulfate;
collagen hydrolysates bearing trimethylammonium chloride and trimethylstearylammonium chloride groups, which are sold under the name Quat-Pro S by the company Maybrook and are referred to in the CTFA dictionary as Steartrimonium Hydrolyzed Collagen;
animal protein hydrolysates which bear trimethylbenzylammonium groups, such as the products sold under the name Crotein BTA by the company Croda and referred to in the CTFA dictionary as Benzyltrimonium hydrolyzed animal protein;
protein hydrolysates bearing quaternary ammonium groups on the polypeptide chain, said ammonium groups containing at least one alkyl radical having from 1 to 18 carbon atoms.

Among these protein hydrolysates, mention may be made, inter alia, of:
Croquat L, in which the quaternary ammonium groups contain a C₁₂ alkyl group;
Croquat M, in which the quaternary ammonium groups contain C₁₀-C₁₈ alkyl groups;
Croquat S, in which the quaternary ammonium groups contain a C₁₈ alkyl group;
Crotein Q, in which the quaternary ammonium groups contain at least one alkyl group having from 1 to 18 carbon atoms.

These various products are sold by the company Croda.

Other quaternized proteins or hydrolysates are, for example, those corresponding to the formula (XV):

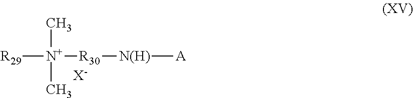

in which X⁻ is an anion of an organic or inorganic acid, A denotes a protein residue derived from collagen protein hydrolysates, R₂₉ denotes a lipophilic group comprising up to 30 carbon atoms, R₃₀ represents an alkylene group having 1 to 6 carbon atoms. Mention may be made, for example, of the products sold by the company Inolex, under the name Lexein QX 3000, referred to, in the CTFA dictionary, as Cocotrimonium Collagen Hydrolysate.

Mention may also be made of quaternized plant proteins such as wheat, corn or soybean proteins: quaternized wheat proteins that may be mentioned include those sold by the company Croda under the names Hydrotriticum WQ or QM, which in the CTFA dictionary are called Cocodimonium Hydrolysed wheat protein, or Hydrotriticum QL, which in the CTFA dictionary is called Laurdimonium hydrolysed wheat protein, or else Hydrotriticum QS, which in the CTFA dictionary is called Steardimonium hydrolysed wheat protein.

Among all the cationic polymers that may be used in the context of the present invention, it is preferred to use the compounds of families (1), (6), (7) and (8) and in particular of families (1), (6) and (7), and mixtures thereof.

The cationic polymer(s) ii) may be present in an amount ranging from 0.01% to 10% by weight, even better still between 0.1% and 8% by weight, and even more preferentially from 0.5% to 5% by weight, relative to the total weight of the composition containing it or them.

Preferably, the composition of step (3) comprises at least one cationic conditioning agent.

According to one particular embodiment, the composition comprising the hair treatment agent(s), after a leave-on time of generally less than 20 min, preferably ranging from 1 to 15 min, is rinsed off before the heat application step is carried out.

The compositions used in the method of the invention, and in particular the one containing glyoxylic acid and/or derivatives thereof, may also comprise at least one common cosmetic ingredient, chosen in particular from propellants; oils; non-liquid fatty substances and in particular C₈-C₄₀ esters, C₈-C₄₀ acids; surfactants; sunscreens; moisturizers; antidandruff agents; antioxidants; chelating agents; nacreous agents and opacifiers; plasticizers or coalescers; fillers; silicones and in particular polydimethylsiloxanes; polymeric or non-polymeric thickeners; gelling agents; emulsifiers; conditioning or styling polymers; fragrances; basifying agents or acidifying agents; silanes; crosslinking agents. The composition can, of course, comprise several cosmetic ingredients appearing in the above list.

These compositions can also comprise one or more organic solvents which are in particular water-soluble, such as C₁-C₇ alcohols; mention may in particular be made of C₁-C₇ aliphatic or C₆-C₇ aromatic monoalcohols, and C₃-C₇ polyols and polyol ethers, which can be used alone or as a mixture with water.

Depending on their nature and the purpose of the compositions, the usual cosmetic ingredients may be present in usual amounts which can be easily determined by those skilled in the art and which can be, for each ingredient, between 0.01% and 80% by weight. Those skilled in the art will take care to choose the ingredients included in the composition, and also the amounts thereof, such that they do not harm the properties of the compositions which are of use in the present invention.

According to one particular embodiment, the composition containing glyoxylic acid and/or a derivative thereof comprises at least one nonionic cellulose-based polymer.

According to the invention, the term “cellulose-based” polymer is intended to mean any polysaccharide compound which has in its structure sequences of glucose residues connected by β-1,4 bonds. Thus, the cellulose-based polymers of the invention may be chosen from unsubstituted celluloses, including in a microcrystalline form, and cellulose ethers. Among these cellulose-based polymers, cellulose ethers, cellulose esters and cellulose ester ethers are distinguished.

By way of example of nonionic cellulose-based polymers, mention may be made of (C₁-C₄)alkylcelluloses, such as methylcelluloses and ethylcelluloses (for example, Ethocel standard 100 Premium from Dow Chemical); (poly)hydroxy(C₁-C₄)alkylcelluloses, such as hydroxymethylcelluloses, hydroxyethylcelluloses (for example, Natrosol 250 HHR provided by Aqualon) and hydroxypropylcelluloses (for example, Klucel EF from Aqualon); mixed (poly)hydroxy(C₁-C₄)alkyl(C₁-C₄)alkylcelluloses, such as hydroxypropylmethylcelluloses (for example, Methocel E4M from Dow Chemical), hydroxyethylmethylcelluloses, hydroxyethylethylcelluloses (for example, Bermocoll E 481 FQ from Akzo Nobel) and hydroxybutylmethylcelluloses.

According to one particular embodiment, the cellulose-based polymers are nonionic cellulose-based polymers comprising alkyl chains comprising from 1 to 6 carbon atoms. Preferably, the cellulose-based polymer(s) of the invention are chosen from cellulose ethers, in particular hydroxyalkylalkylcelluloses, such as (poly)hydroxy(C₁-C₄)alkyl(C₁-C₄)alkylcelluloses, in particular hydroxypropylméthylcelluloses (for example, Methocel F4M from Dow Chemical), hydroxyethylmethylcelluloses, hydroxyethylethylcelluloses (for example, Bermocoll E 481 FQ from Akzo Nobel) and hydroxybutylmethylcelluloses.

The preferred cellulose-based polymers are cellulose-based polymers which do not comprise a fatty chain, i.e. which preferably do not comprise a chain comprising more than 10 carbon atoms.

The cellulose-based polymer(s) can be present in the composition containing glyoxylic acid and/or a derivative thereof in contents ranging from 0.05% to 10% by weight, in particular from 0.1% to 5% by weight and even better still from 0.1% to 3% by weight, relative to the total weight of the composition.

After the composition applied in step (ii) has been rinsed off, the method of the invention then comprises a heat application step comprising at least one step of heating at a temperature of at least 150° C.

According to one embodiment, the heating at a temperature of at least 150° C. is carried out using a straightening iron. Straightening with an iron is known from the prior art. It consists in straightening the hair with flat heating tongs, which are generally metallic. The straightening irons are generally used at a temperature of at least 150° C., preferably ranging from 200 to 250° C.

This step of straightening by application of heat can be carried out in two steps: a first step which consists in straightening the hair by means of a brush and a hairdryer (step known as blow drying). This blow drying step is generally carried out at between 40 and 70° C.; a second step which consists in straightening the hair with a straightening iron as previously described.

According to one particular embodiment, the method of the invention comprises the application of the composition containing glyoxylic acid and/or derivatives thereof to dry hair, a contact time of the composition on the hair ranging from 10 to 60 minutes, preferably from 20 to 40 minutes, the application, without rinsing, of a composition comprising at least one compound chosen from fatty alcohols, cationic polymers and cationic surfactants, then, optionally after rinsing, the straightening of the hair with a brush and a hairdryer (blow drying), followed by straightening of the hair with a straightening iron at a temperature of at least 150° C., preferably ranging from 200 to 250° C., preferably 200 to 230° C.

According to one variant of a method of the invention, the application of the composition containing glyoxylic acid and/or derivative thereof is carried out on hair shampooed beforehand. The shampoo used in the method of the invention preferably comprises at least one amphoteric surfactant optionally combined with at least one cationic surfactant. According to one particular embodiment, the shampoo used does not contain anionic surfactants.

The amphoteric surfactants which can be used are, for example, betaine surfactants, such as cocobetaine or cocoamidopropylbetaine or the compounds classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

According to this embodiment, the hair is rinsed and preferably pre-dried (total drying or partial drying with a hairdryer or with a towel) before the application of the composition comprising glyoxylic acid and/or at least one derivative thereof.

According to one variant, the method of the invention may comprise the application of other hair agents as a pre-treatment or post-treatment to steps (i) and (iii). Preferably, the method of the invention comprises, after the washing of the hair and before the application of the composition containing glyoxylic acid and/or at least one derivative thereof, a pretreatment which comprises the application to the hair, preferably to wet hair, of a composition comprising at least one siliceous compound comprising at least one silicon atom, such as an alkoxysilane which is optionally organically modified, in particular with one or more amino groups, or a silicone, for example a polydimethylsiloxane which is optionally substituted, in particular with one or more amino groups. By way of example of siliceous compounds, mention may be made of aminopropyltriethyloxysilane and amodimethicones. The application of this pretreatment is preferably not rinsed off before the application of the composition containing glyoxylic acid and/or at least one derivative thereof, and is preferably followed by pre-drying (total drying or partial drying with a hairdryer or with a towel) before the application of the composition containing glyoxylic acid and/or at least one derivative thereof.

According to one particular embodiment, the straightening with the straightening iron is performed in several passes on the hair, in general 8 to 10 passes.

### EXAMPLE

The composition comprising glyoxylic acid described below is applied to naturally frizzy hair having been previously washed, rinsed and then pre-dried;

the contents are indicated as crude starting material (commercial product as it is).

| | Content |
|---|---|
| Chemical name | (g) |
| LACTIC ACID | 2.5 |
| GLYOXYLIC ACID IN AQUEOUS | 16 |
| SOLUTION AT 50% | |
| HYDROXYPROPYL- | 1 |
| METHYLCELLULOSE (METHOCEL | |
| F 4 M from DOW CHEMICAL) | |
| CROSSLINKED | 2.5 |
| ETHYLTRIMETHYLAMMONIUM | |
| METHACRYLATE CHLORIDE | |
| HOMOPOLYMER, AS | |
| A DISPERSION IN A MIX OF | |
| ESTERS AT 50%; | |
| (SALCARE SC 96 from BASF) | |
| AMINO MODIFIED SILICONE- | 2 |
| POLYETHER | |
| COPOLYMER (SILSOFT A+ | |
| from MOMENTIVE | |
| PERFORMANCE MATERIALS) | |
| SODIUM N-COCOYL | 2 |
| AMIDOETHYL-N- | |
| ETHOXYCARBOXYMETHYL | |
| GLYCINATE IN | |
| AQUEOUS SOLUTION AT 31.5% | |
| (MIRANOL C2M | |
| CONC NP from RHODIA) | |
| SODIUM HYDROXIDE | qs pH 2.2 |
| DEIONIZED WATER | qs 100 |

Without rinsing off this composition comprising glyoxylic acid, and after a leave-on time of 20 min, the following composition is applied:

| | Content |
|---|---|
| Chemical name | (g) |
| LACTIC ACID | 0.1 |
| CITRIC ACID | 0.3 |
| LIQUID PETROLEUM JELLY (MARCOL | 3 |
| 82 from EXXONMOBIL CHEMICAL) | |
| CETYLSTEARYL ALCOHOL | 9 |
| (C16/C18 50/50) (NAFOL 1618 | |
| EN from SASOL) | |
| MYRISTYL/CETYL/STEARYL | 1 |
| MYRISTATE/PALMITATE/ | |
| STEARATE MIXTURE | |
| (CRODAMOL MS-PA-(MH) from CRODA) | |
| CETYLTRIMETHYLAMMONIUM | 3.2 |
| CHLORIDE IN AQUEOUS | |
| SOLUTION (GENAMIN CTAC | |
| 25 from CLARIANT) | |
| DIPALMITOYLETHYLHYDROXY- | 4.5 |
| ETHYLMETHYLAMMONIUM | |
| METHOSULFATE (30)/ | |
| CETEARYL ALCOHOL (70) | |
| (DEHYQUART F 30 from COGNIS) | |
| Preserving agents | qs |

After a waiting time of 10 min, the hair is rinsed, blow-dried, and then straightened with a straightening iron. Relaxed, straightened hair with a good cosmetic feel and without the secondary drawbacks associated with the application is thus obtained.

### Example 2

Improved results are obtained by using, as pretreatment on the hair initially washed, a 50/50 mixture of the two compositions A and B below containing 3-aminopropyltriethoxysilane as siliceous compound, and then by applying, without rinsing but with pre-drying, the method described in example 1 starting from the application of the composition with glyoxylic acid.

| | Composition | Composition |
|---|---|---|
| | A (in g) | B (in g) |
| HYDROXYETHYL CELLULOSE (MW: 1 | 1 | — |
| 300 000) (NATROSOL 250 HHR PC | | |
| from ASHLAND) | | |
| ALPHA,OMEGA-DIHYDROXYLATED | — | 65 |
| POLYDIMETHYLSILOXANE/ | | |
| CYCLOPENTADIMETHYLSILOXANE | | |
| MIXTURE (14.7/85.3) (XIAMETER | | |
| PMX-1501 FLUID from DOW CORNING) | | |
| 3-AMINOPROPYLTRIETHOXYSILANE | 1 | |
| (SILSOFT A-1100 from MOMENTIVE | | |
| PERFORMANCE MATERIALS) | | |
| ABSOLUTE ETHYL ALCOHOL | | 5 |
| PRESERVING AGENTS | qs | — |
| CASTOR OIL WITH 40 OE | 2.4 | — |
| (CREMOPHOR CO 40 SURFACTANT | | |
| from BASF) | | |
| LACTIC ACID | 0.25 | — |
| DEIONIZED WATER | qs 100 | — |
| CYCLOPENTADIMETHYLSILOXANE | | qs 100 |

1. A method for straightening/relaxing keratin fibers, in particular the hair, which comprises (i) the application to said fibers of a cosmetic composition comprising at least glyoxylic acid and/or at least one derivative thereof, the composition having a pH of less than or equal to 4, (ii) a leave-on time for the composition comprising glyoxylic acid and/or at least one derivative thereof of at least 10 min, followed by (iii) the application, without rinsing, of a composition comprising at least one hair treatment compound chosen from fatty alcohols and cationic conditioning agents, and then by (iv) a heat application step comprising at least one step of heating at a temperature of at least 150° C.

2. The method as claimed in claim 1, wherein the glyoxylic acid and/or derivatives thereof is in free form or in hydrate form or in salt form.

3. The method as claimed in claim 1, wherein the glyoxylic acid derivatives are chosen from glyoxylic acid esters, glyoxylic acid amides and glyoxylic acid acetals and hemiacetals.

4. The method as claimed in claim 1, wherein the composition of step (i) comprises glyoxylic acid in free form or in hydrate form.

5. The method as claimed in any one of the preceding claims, wherein the amount of glyoxylic acid and/or of derivatives thereof is at least 3% by weight of the total weight of the composition, preferably ranging from 3% to 10% by weight of the total weight of the composition.

6. The method as claimed in any one of the preceding claims, wherein the composition containing glyoxylic acid and/or a derivative thereof additionally comprises a nonionic cellulose-based polymer.

7. The method as claimed in any one of the preceding claims, wherein, in step (ii), the leave-on time ranges from 10 to 60 min, preferably from 20 to 35 min.

8. The method as claimed in any one of the preceding claims, wherein, in step (iii), at least one hair treatment compound chosen from fatty alcohols and/or cationic polymers and/or cationic surfactants is applied.

9. The method as claimed in claim 8, wherein the conditioning compound ? chosen from fatty alcohols and/or cationic polymers and/or cationic surfactants is included in a composition free of glyoxylic acid and/or a derivative thereof.

10. The method as claimed in any one of the preceding claims, wherein the hair treatment compound(s) chosen from fatty alcohols and/or cationic conditioning agents is (are) present for each of the categories in an amount ranging from 0.01% to 10% by weight, even better still from 0.1% to 8% by weight and even more preferentially from 0.5% to 5% by weight relative to the total weight of the composition containing it or them.

11. The method as claimed in any one of the preceding claims, wherein the composition (iii) containing the hair treatment compound(s) chosen from fatty alcohols and/or cationic conditioning agents, after a leave-on time of generally less than 20 min, preferably ranging from 1 to 15 min, is rinsed off before the heat application step is carried out.

12. The method as claimed in any one of the preceding claims, wherein the heat application step comprises a single step which consists in straightening the hair with a straightening iron at a temperature of at least 150° C., preferably from 200 to 250° C.

13. The method as claimed in any one of the preceding claims, wherein the heat application step comprises a first step which consists in straightening the hair by means of a brush and a hairdryer (step known as blow drying) and a second step which consists in straightening the hair with a straightening iron at a temperature of at least 150° C., preferably from 200 to 250° C.

14. The method as claimed in any one of the preceding claims, comprising, before the application of the composition of step (i), a pretreatment which comprises the application of a composition comprising at least one siliceous compound comprising at least one silicon atom, preferably an alkoxysilane optionally organically modified with one or more amino groups, or a silicone, preferably a polydimethylsiloxane optionally substituted with one or more amino groups.

15. The method as claimed in the preceding claim, wherein the pretreatment composition comprising at least one siliceous compound is not rinsed off.
